(19) United States
(12) Patent Application Publication
(10) Pub. No.: US 2025/0009982 A1
(43) Pub. Date: Jan. 9, 2025

(54) RETRACTABLE PREFILLED SYRINGE WITH COLLAPSIBLE PLUNGER

(71) Applicant: RONCADELLE OPERATIONS SRL, Castel Mella (Bs) (IT)

(72) Inventor: Erik RYCKALTS, Castel Mella (Bs) (IT)

(21) Appl. No.: 18/705,686

(22) PCT Filed: Nov. 2, 2022
(86) PCT No.: PCT/EP2022/080484

§ 371 Date: Apr. 29, 2024

(30) Foreign Application Priority Data
Nov. 2, 2021 (EP) 21205847.3

(51) Int. Cl. A61M 5/32; A61M 5/20; A61M 5/315

(52) U.S. Cl. CPC A61M 5/3234; A61M 5/31505; A61M 5/3221; A61M 2005/2006

(57) ABSTRACT

The current invention relates to a syringe assembly using prefilled syringes provided with or without plunger. The syringe assembly enables the safe use of said syringes by including a syringe retraction mechanism, which mechanism is automatically actuated after an injection. Upon retraction the syringe is maintained enclosed and without possibility of being exposed. When active, the retraction mechanism causes the syringe assembly to have a compact envelope which does not leave the user in doubt about the state of the assembly.

### FIELD OF THE INVENTION

The present invention relates to an injection device. In particular, the present invention relates to a safety syringe application to prefilled syringes.

### BACKGROUND

Many attempts have been made to provide for retractable syringes assemblies, in particular, assemblies which make use of pre-filled syringes of the type traditionally provided in a glass ampoule or the like.

U.S. Pat. No. 4,624,660 discloses a retractable needle arrangement for a glass ampoule. Other examples include U.S. Pat. Nos. 5,624,400 and 5,634,909. These applications are amongst many that provide for ampoule based retractable needle operation where a standard ampoule is provided and retractable needle operations are carried out around the standard ampoule.

AU2011265487 describes a syringe assembly for a prefilled glass syringe, which assembly, upon finishing an injection, permits retracting of said glass syringe into a sleeve formed by an extension of the outer barrel of the syringe assembly. While the assembly disclosed in this document permits the retraction of the syringe, and consequently, the needle of said syringe, the retraction only happens when the user releases the plunger. This results in the needle being exposed long after the injection takes place and requires the user to regrasp the syringe assembly. Furthermore, the state of the assembly, even after retraction of the syringe may leave the user in doubt as to whether the syringe has been used or not.

FR2799976A1, in the name of Plastic Omnium SA, describes a syringe comprising a barrel with a needle holder and plunger. The syringe has a sleeve in which the barrel can slide axially between an injection and retracted positions. While the device of FR '976 permits covering the needle of the syringe, it does not render the plunger of the syringe inoperable.

GB2404338A, in the name of Safe T Ltd., describes a actuator and containment device for a syringe. The device comprises a hollow body, a spring and a plunger, in which respect the hollow body has internal latching formations and is adapted to house the syringe barrel and piston means. The device of GB '338 disadvantageously requires said hollow body, and in particular the latching formations to be adapted to each specific type of syringe piston. A similar device is disclosed in U.S. Pat. No. 5,330,430A, in the name of Robert J. Sullivan. The device of US '430, a retractable syringe applicator, disadvantageously requires adaptation of the plunger of the syringe in order to be operable.

The aim of the invention is to provide a method which eliminates those disadvantages.

### SUMMARY OF THE INVENTION

The present invention and embodiments thereof serve to provide a solution to one or more of above-mentioned disadvantages. To this end, the present invention relates to syringe assembly according to claim 1.

Preferred embodiments of the device are shown in any of the claims 2 to 15. The syringe assembly disclosed in this claim provides an advantageously safe way to administer injectable medicinal products provided in a prefilled syringe.

A specific preferred embodiment relates to an invention according to claim 11. This preferred embodiment of the syringe assembly permits the use of prefilled syringes, which syringes do not include a plunger.

A specific preferred embodiment relates to an invention according to claim 13. This preferred embodiment permits the use of prefilled syringes, which syringes already include a plunger.

The syringe assembly disclosed in the claims allows the safe use of prefilled syringes by providing a retraction mechanism, which mechanism retracts and completely encloses said syringes inside the body of the assembly. Furthermore, the mechanism of the syringe assembly is such that it does not leave the user in doubt regarding the state of the syringe assembly.

### DESCRIPTION OF FIGURES

The following description of the figures of specific embodiments of the invention is merely exemplary in nature and is not intended to limit the present teachings, their application or uses. Throughout the drawings, corresponding reference numerals indicate like or corresponding parts and features.

FIG. 1 shows a first embodiment of the syringe assembly wherein the retraction piece includes an inner plunger, shown in the figure in a pre-injection state.

FIG. 2 shows the first embodiment of the syringe assembly wherein the retraction piece includes an inner plunger, shown in the figure in a pre-retraction state.

FIG. 3 shows the first embodiment of the syringe assembly wherein the retraction piece includes an inner plunger, shown in the figure in a retracted state.

FIG. 4 shows a second embodiment of the syringe assembly where the retraction piece attaches to the plunger of a prefilled syringe.

FIG. 5 shows the a detailed view of the retraction piece of the second embodiment.

### DETAILED DESCRIPTION OF THE INVENTION

The present invention concerns a syringe assembly.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the invention provides a syringe assembly 1 comprising:
a. an outer barrel 2 having a hollow first proximal section suitable to receive a radially extending lip 16 of a prefilled syringe 6, said first section being in communication with a hollow second section with a smaller diameter, said second section being suitable to receive the barrel of a prefilled syringe 6;
b. a retraction piece 5 comprising a first radially extending lip 8, and at least a second radially extending lip, the retraction piece 5 further comprising a rear extension 9 extending rearwards from the proximal end of the retraction piece 5.
c. an outer plunger 3 having a first proximal hollow cavity and a second distal cavity, said second hollow cavity being of substantially larger diameter than the first hollow cavity, said second hollow cavity being suitable to receive the retraction piece 5;
d. a spring support cap 13 having a proximal end equipped with a radially extending flange and an intermediate section in rigid connection with the proximal end of the outer plunger 3, the spring support cap 13 being further equipped with an extension 4 at its distal end; and
e. a spring 7 having a first proximal end and a second distal end, said first end being rigidly attached to the distal end of the spring support cap 13 and said second end being in rigid connection with the rear extension 9 of the retraction piece 5;

The outer plunger 3 has a rear position A and a forward position B, in which position A the retraction piece 5 is held in an advanced position relative to the outer plunger 3 by a retention element 12, and in which position B the retraction piece 5 is caused to override said retention feature and retract into the outer plunger 3 under action of the spring 7. By preference, the spring 7 is a tension spring. More preferably, position A is adaptable to any pre-set injection volume of the prefilled syringe.

In a preferred embodiment, the retention element is an annular ring 12 extending from the inner surface of the second section of the outer plunger 3. By preference, the annular ring 12 has a smaller inner diameter than the outer diameter of the radially extending lips 8 of the retraction piece 5. This permits opposing the retraction of the retraction piece 5 under the action of the spring 7 until the outer plunger 3 is in position B.

In a further or another embodiment, the first section of the outer barrel 2 is equipped with at least two bendable tabs 14 extending towards the distal end and towards the longitudinal axis of the outer barrel 2, said bendable tabs 14 being located near the distal end of the first section of the outer barrel 2. This permits easy installation of a prefilled syringe 6 inside the outer barrel 2. Furthermore, the bendable tabs 14 permit retaining the prefilled syringe 6 in place until the end of the injection.

In a further or another embodiment, at least part of the transition of the first section of the outer barrel 2 to the second section of the outer barrel 2 forms an abutment surface 15 substantially perpendicular to the longitudinal axis of the outer barrel 2. This abutment 15 advantageously supports the lip 16 of the prefilled syringe 6, thus permitting a stable positioning and retention of the syringe 6 within the outer barrel 2, before and during injection.

In a further or another embodiment, the outer plunger 3 is further equipped with an annular groove 10 near its distal end, which groove is complimentary to the bendable tabs 14 of the outer barrel 2 when the outer plunger is in position B. The annular groove 10 advantageously permits retaining the outer plunger 3 in advanced position B under the opposition of the bendable tabs 14 of the outer barrel 2. In this way, the syringe assembly 1 presents a smaller envelope after injection, which smaller envelope advantageously facilitates storage and disposal of used syringe assemblies 1. This smaller envelope also informs the any user that the syringe assembly 1 has been used and is empty of its medicinal contents, thereby not leaving the user in doubt regarding the state of the syringe assembly 1.

In a further or another embodiment, the distance between the abutment 15 and the distal end of the outer barrel 2 is at least as long as the barrel of the prefilled syringe 6. In this way the full length of the barrel of the syringe 6 remains supported and protected before and during the injection. More importantly, the distance between the abutment 15 and the distal end of the outer barrel 2 provide a protective sleeve that fully encases the needle of the syringe 6 after the injection. In this way, the needle cannot come in contact with the user once the injection is concluded. More preferably, the distance between the abutment 15 and the distal end of the outer barrel 3 extends past the barrel of the syringe such that a syringe cap can be partially supported by the distal end of the outer barrel 2.

In a further or another embodiment, the rear extension 9 of the retraction piece 5 comprises at least two hooking tabs. In this way, assembly and retention of the distal end of the spring 7 are improved, which advantageously assures proper retraction and retention of the prefilled syringe 6 after injection.

In a further or another embodiment, the second hollow cavity of the outer plunger 3 has an internal diameter larger than the diameter of the radially extending lip 16 of a prefilled syringe 6. This permits the retraction of the retraction piece 5 and syringe 6 into the body of the outer plunger 3. In this way, the overall length of the syringe assembly 1 is kept advantageously compact, which permits better handling of the syringe assembly 1, before, during and after injection. Furthermore, the compact size of the syringe assembly 1 facilitate storage both before and after use as well as disposal of the syringe assembly 1 after use.

In a further or another embodiment, the bendable tabs of the outer barrel are bendable between 30° and −15°. This permits sufficient bending amplitude to engage both the lip 16 of a prefilled syringe 6 as well as sufficient retention of the outer plunger 3 by acting on its groove 10.

In a further or another embodiment, the retraction piece 5 further comprises an inner plunger 11. By preference, the diameter of the inner plunger 11 is smaller than the inner diameter of the barrel of a prefilled syringe 6. More preferably, the inner plunger 11 attaches to the stopper 20 of a prefilled syringe 6 upon being pressed or twisted against said rear seal. In this way, the retraction force exerted by the spring 7 upon release of the retraction piece 5 from the outer plunger bendable tabs 14 is advantageously transmitted to the syringe 6 by means of friction between the stopper 20 and the inner surface of the syringe 6. This permits the use and retraction of prefilled syringes which are provided without a plunger.

In a further or another embodiment, the retraction piece 5 has a hollow cavity suitable to receive the proximal end of a plunger 19 of a prefilled syringe 6. By preference, the retraction piece 5 is further equipped with at least two bendable tabs 18. More preferably, the tabs of the retraction piece are bendable between −30° and 15°. In this way, the proximal end of the plunger 19 is securely held by the retraction piece 5 thereby allowing force exerted by spring 7 to act be transmitted to the syringe 6 by means of friction between the stopper 20 and the inner surface of the syringe 6 once the retraction piece 5 is released. This advantageously permits the use prefilled syringes having a plunger while ensuring effective retraction of the syringe 6 after the injection.

However, it is obvious that the invention is not limited to syringes for administering medicinal products. The syringe assembly according to the invention can be applied in all sorts of injectable fluids.

The invention is further described by the following non-limiting drawings which further illustrate the invention, and are not intended to, nor should they be interpreted to, limit the scope of the invention.

### DESCRIPTION OF FIGURES

With as a goal illustrating better the properties of the invention the following presents, as an example and limiting in no way other potential applications, a description of a number of preferred embodiments of a syringe assembly based on the invention, wherein:

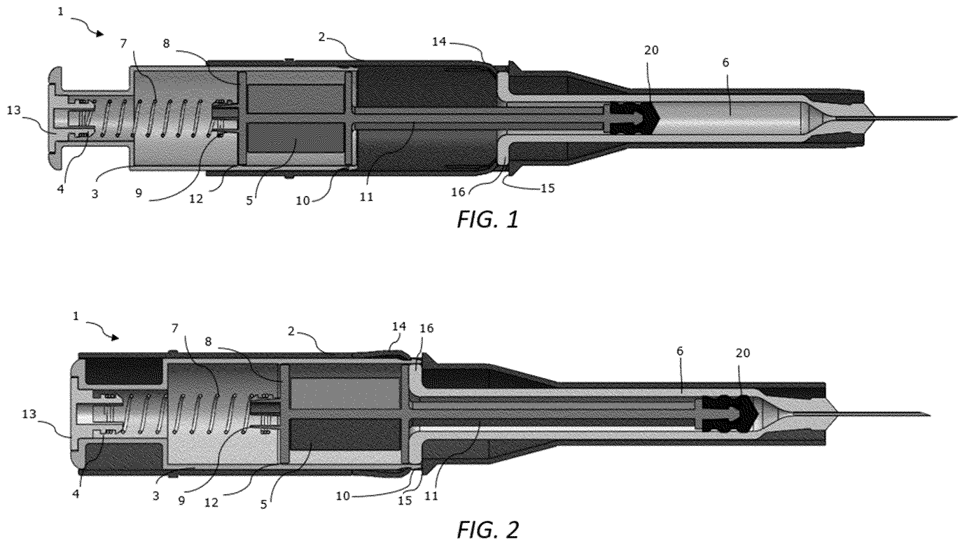

FIG. 1 shows a first embodiment of the syringe assembly 1 wherein the retraction piece 5 includes an inner plunger 11, shown in the figure in a pre-injection state. The syringe assembly 1 includes a prefilled syringe 6, which syringe is held inside the outer barrel 2 by means of a pair of bendable tabs 14 and an abutment 15 acting upon the syringe lip 16. The inner plunger 11 extending from the retraction piece 5 is shown inserted into the barrel of the syringe 6 and attached to stopper 20. A spring 7 is shown in its extended state, which spring 7 is attached to the retraction piece extension 9 and to the spring support cap extension 4 extending from the spring support cap 13. The retraction piece 5 is prevented from retracting under the action of the spring 7 by the interference between the outer plunger ring 12 and the retraction piece lip 8. The outer plunger 3 is shown provided with a grove 10.

FIG. 2 shows the first embodiment of the syringe assembly 1 wherein the retraction piece 5 includes an inner plunger 11, shown in the figure in a pre-retraction state. The syringe assembly 1 including a prefilled syringe 6, which syringe remains in its advanced position and released from the outer barrel tabs 14 by intermediate of the outer plunger 3, which outer plunger 3 now encases the syringe lip 16. The inner plunger 11 extending from the retraction piece 5 is shown fully inserted into the barrel of the syringe 6 and attached to stopper 20. The spring 7 is shown still in its extended state, which spring 7 is attached to the retraction piece extension 9 and to the spring support cap extension 4 extending from the spring support cap 13. The retraction piece 5 is still in its forward position and being prevented from retracting under the action of the spring 7 by the interference between the outer plunger ring 12 and the retraction piece lip 8. The outer plunger 3 is shown almost in advanced position B, the outer plunger groove 10 being shown engaged by the outer barrel bendable tabs 14.

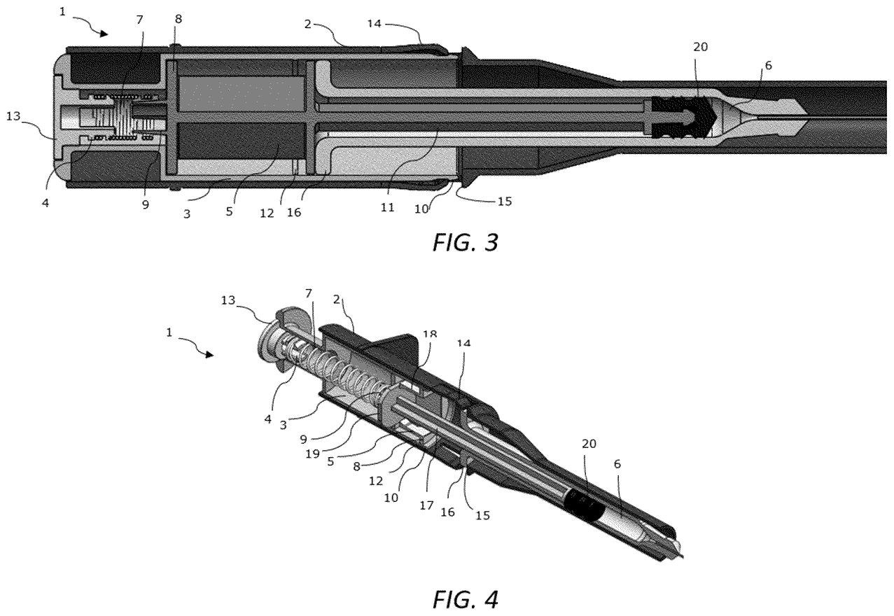

FIG. 3 shows the first embodiment of the syringe assembly 1 wherein the retraction piece 5 includes an inner plunger 11 shown in the figure in a retracted state. The syringe assembly 1 including a prefilled syringe 6, into which syringe the inner plunger 11 is shown fully inserted, which plunger 11 is shown attached to stopper 20. Spring 7 is shown in a contracted state, which spring 7 is shown holding the retraction piece 5, the inner plunger 11 and the syringe 6 in their retracted position inside the outer barrel 2. The outer plunger 3 is shown in advanced position B, the outer plunger groove 10 being shown engaged by the outer barrel bendable tabs 14.

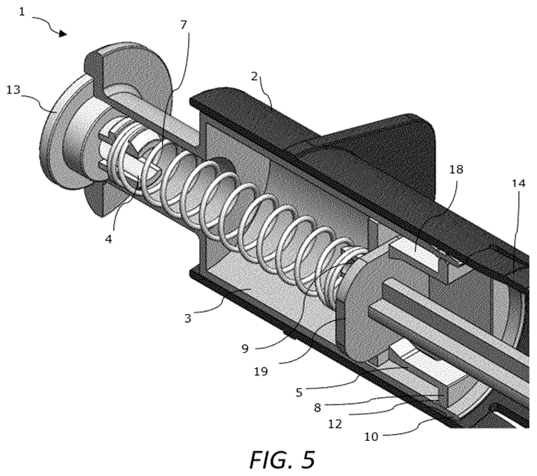

FIG. 4 and FIG. 5 show a second embodiment of the syringe assembly 1 where the retraction piece attaches to the plunger of a prefilled syringe. FIG. 4 shows a prefilled syringe 6 with its own plunger 17 with an integral stopper 20 inside the outer barrel 2, which syringe is retained by a pair of bendable tabs 14 acting against the lip 16 of the syringe, keeping said lip 16 resting against abutment 15. In FIG. 4 and FIG. 5, the outer plunger 3 is shown in its rear position A inside the outer barrel 2 and equipped with a spring support cap 13, which spring support cap 13 is shown provided with an extension 4 supporting the proximal end of spring 7. The spring 7 is shown in its extended position and with its distal end attached to the retraction piece extension 9, thereby pulling back the retraction piece 5, which rearward motion is prevented by the interference between the retraction piece lip 8 and the outer plunger ring. The proximal end of the syringe plunger 19 is shown inserted through the distal end of the retraction piece 5 and retained therein by means of two bendable tabs 18.

### LIST OF NUMBERED ITEMS

1 Syringe assembly
2 outer barrel
3 outer plunger
4 spring support cap extension
5 retraction piece
6 prefilled syringe
7 spring
8 retraction piece lip
9 retraction piece extension
10 outer plunger groove
11 inner plunger
12 outer plunger ring
13 spring support cap
14 outer plunger bendable tab
15 abutment
16 syringe lip
17 syringe plunger
18 retraction piece bendable tab
19 proximal end of syringe plunger
20 stopper

The present invention is in no way limited to the embodiments described in the examples and/or shown in the figures. On the contrary, assemblies according to the present invention may be realized in many different ways without departing from the scope of the invention.

1. A syringe assembly comprising:
an outer barrel having a hollow first proximal section suitable to receive a radially extending lip of a prefilled syringe, said first proximal section being in communication with a hollow second section with a smaller diameter, said second section being suitable to receive the outer barrel of a prefilled syringe;
a retraction piece comprising a first radially extending lip, and at least a second radially extending lip, the retraction piece further comprising a rear extension extending rearwards from a proximal end of the retraction piece;
an outer plunger having a first proximal hollow cavity and a second distal hollow cavity, said second distal hollow cavity being of substantially larger diameter than the first hollow cavity, said second distal hollow cavity being suitable to receive the retraction piece;
a spring support cap having a proximal end equipped with a radially extending flange and an intermediate section in rigid connection with the proximal end of the outer plunger, the spring support cap being further equipped with an extension at its distal end; and
a spring having a first proximal end and a second distal end, said first end being rigidly attached to the distal end of the spring support cap and said second end being in rigid connection with the rear extension of the retraction piece;
characterized in that, the outer plunger has a rear position A and a forward position B, in which position A the retraction piece is held in an advanced position relative to the outer plunger by a retention element, and in which position B the retraction piece is caused to override said retention element and retract into the outer plunger under action of the spring.

2. The syringe assembly according to claim 1, characterized in that, the retention element is an annular ring extending from an inner surface of the second section of the outer plunger.

3. The syringe assembly according to claim 2, characterized in that, the annular ring has a smaller inner diameter than the outer diameter of the radially extending lips of the retraction piece.

4. The syringe assembly according to claim 1, characterized in that, the first section of the outer barrel is equipped with at least two bendable tabs extending towards the distal end and towards a longitudinal axis of the outer barrel, said bendable tabs being located near the distal end of the first section of the outer barrel.

5. The syringe assembly according to claim 4, characterized in that, at least part of a transition of the first section of the outer barrel to the second section of the outer barrel forms an abutment surface substantially perpendicular to the longitudinal axis of the outer barrel.

6. The syringe assembly according to claim 4, characterized in that, the outer plunger is further equipped with an annular groove near its distal end, which groove is complimentary to the bendable tabs of the outer barrel when the outer plunger is in position B.

7. The syringe assembly according to claim 5, characterized in that, a distance between the abutment surface and the distal end of the outer barrel is at least as long as the barrel of the prefilled syringe.

8. The syringe assembly according to claim 1, characterized in that, the rear extension of the retraction piece comprises at least two hooking tabs.

9. The syringe assembly according to claim 1, characterized in that, the second hollow cavity of the outer plunger has an internal diameter larger than the diameter of the radially extending lip of a prefilled syringe.

10. The syringe assembly according to claim 4, characterized in that, the bendable tabs of the outer barrel are bendable between 30° and −15°.

11. The syringe assembly according to claim 1, characterized in that, the retraction piece further comprises an inner plunger.

12. The syringe assembly according to claim 11, characterized in that, the diameter of the inner plunger is smaller than the inner diameter of the barrel of a prefilled syringe.

13. The syringe assembly according to claim 1, characterized in that, the retraction piece has a hollow cavity suitable to receive the proximal end of a plunger of a prefilled syringe.

14. The syringe assembly according to claim 13, characterized in that, the retraction piece is further equipped with at least two bendable tabs.

15. The syringe assembly according to claim 10, characterized in that, the bendable tabs of the retraction piece are bendable between −30° and 15°.
